SEQUENCE LISTING (1 sequence)

SEQ ID NO: 1 - protein, 1855 aa, Homo sapiens Homo sapiens
MAASELYTKFARVWIPDPEEVWKSAELLKDYKPGDKVLLLHLEEGKDLEYHLDPKTKELPHLRNPDILVGENDLTALSYLHEPAVLHNLRVRFIDSKLIYTYCGIVLVAINPYEQLPIYGEDIINAYSGQNMGDMDPHIFAVAEEAYKQMARDERNQSIIVSGESGAGKTVSAKYAMRYFATVSGSASEANVEEKVLASNPIMESIGNAKTTRNDNSSRFGKYIEIGFDKRYRIIGANMRTYLLEKSRVVFQAEEERNYHIFYQLCASAKLPEFKMLRLGNADNFNYTKQGGSPVIEGVDDAKEMAHTRQACTLLGISESHQMGIFRILAGILHLGNVGFTSRDADSCTIPPKHEPLCIFCELMGVDYEEMCHWLCHRKLATATETYIKPISKLQATNARDALAKHIYAKLFNWIVDNVNQALHSAVKQHSFIGVLDIYGFETFEINSFEQFCINYANEKLQQQFNMHVFKLEQEEYMKEQIPWTLIDFYDNQPCINLIESKLGILDLLDEECKMPKGTDDTWAQKLYNTHLNKCALFEKPRLSNKAFIIQHFADKVEYQCEGFLEKNKDTVFEEQIKVLKSSKFKMLPELFQDDEKAISPTSATSSGRTPLTRTPAKPTKGRPGQMAKEHKKTVGHQFRNSLHLLMETLNATTPHYVRCIKPNDFKFPFTFDEKRAVQQLRACGVLETIRISAAGFPSRWTYQEFFSRYRVLMKQKDVLSDRKQTCKNVLEKLILDKDKYQFGKTKIFFRAGQVAYLEKLRADKLRAACIRIQKTIRGWLLRKKYLRMRKAAITMQRYVRGYQARCYAKFLRRTKAATIIQKYWRMYVVRRRYKIRRAATIVLQSYLRGFLARNRYRKILREHKAVIIQKRVRGWLARTHYKRSMHAIIYLQCCFRRMMAKRELKKLKIEARSVERYKKLHIGMENKIMQLQRKVDEQNKDYKCLVEKLTNLEGIYNSETEKLRSDLERLQLSEEEAKVATGRVLSLQEEIAKLRKDLEQTRSEKKCIEEHADRYKQETEQLVSNLKEENTLLKQEKEALNHRIVQQAKEMTETMEKKLVEETKQLELDLNDERLRYQNLLNEFSRLEERYDDLKEEMTLMVHVPKPGHKRTDSTHSSNESEYIFSSEIAEMEDIPSRTEEPSEKKVPLDMSLFLKLQKRVTELEQEKQVMQDELDRKEEQVLRSKAKEEERPQIRGAELEYESLKRQELESENKKLKNELNELRKALSEKSAPEVTAPGAPAYRVLMEQLTSVSEELDVRKEEVLILRSQLVSQKEAIQPKDDKNTMTDSTILLEDVQKMKDKGEIAQAYIGLKETNRSSALDYHELNEDGELWLVYEGLKQANRLLESQLQSQKRSHENEAEALRGEIQSLKEENNRQQQLLAQNLQLPPEARIEASLQHEITRLTNENLDLMEQLEKQDKTVRKLKKQLKVFAKKIGELEVGQMENISPGQIIDEPIRPVNIPRKEKDFQGMLEYKKEDEQKLVKNLILELKPRGVAVNLIPGLPAYILFMCVRHADYLNDDQKVRSLLTSTINSIKKVLKKRGDDFETVSFWLSNTCRFLHCLKQYSGEEGFMKHNTSRQNEHCLTNFDLAEYRQVLSDLAIQIYQQLVRVLENILQPMIVSGMLEHETIQGVSGVKPTGLRKRTSSIADEGTYTLDSILRQLNSFHSVMCQHGMDPELIKQVVKQMFYIIGAITLNNLLLRKDMCSWSKGMQIRYNVSQLEEWLRDKNLMNSGAKETLEPLIQAAQLLQVKKKTDDDAEAICSMCNALTTAQIVKVLNLYTPVNEFEERVSVSFIRTIQMRLRDRKDSPQLLMDAKHIFPVTFPFNPSSLALETIQIPASLGLGFISRV

(19) United States
(12) Patent Application Publication
(10) Pub. No.: US 2021/0063415 A1
(43) Pub. Date: Mar. 4, 2021

(54) APPLICATIONS OF BIOMARKER IN TERMS OF DETERMINING ANTIPHOSPHOLIPID SYNDROME AND REAGENT KIT OF BIOMARKER

(71) Applicant: GUANGZHOU KANGRUN BIOTECHNOLOGY CO., LTD., Guangzhou, Guangdong (CN)

(72) Inventors: Chengde YANG, Guangzhou (CN); Hui SHI, Guangzhou (CN); Wenxi HUANG, Guangzhou (CN)

(21) Appl. No.: 16/961,263

(22) PCT Filed: Jul. 10, 2018
(86) PCT No.: PCT/CN2018/095067

§ 371 Date: Jul. 10, 2020

(30) Foreign Application Priority Data
Jan. 12, 2018 (CN) 201810030852.3

(51) Int. Cl. G01N 33/68; G01N 21/76

(52) U.S. Cl. CPC G01N 33/6893; G01N 2800/32; G01N 33/6854; G01N 21/76

(57) ABSTRACT

Applications of antibodies to MYO5A protein as a biomarker in terms of determining antiphospholipid syndrome. Antibodies to the MYO5A protein include at least one of IgG, IgM or IgA. It can be used to identify primary and secondary antiphospholipid syndrome. At the same time, a reagent kit is provided, which is made of MYO5A protein, to determine whether there is antiphospholipid syndrome by detecting the presence of antibodies to MYO5A protein. The present disclosure, applications of antibodies to MYO5A protein as a biomarker in terms of determining antiphospholipid syndrome, provides a new biomarker to identify antiphospholipid syndrome and a new application direction which is of great significance for the identification of antiphospholipid syndrome.

### TECHNICAL FIELD

The present disclosure relates to the technical field of immunization, and in particular to applications of antibodies to MYO5A protein as a biomarker in terms of determining antiphospholipid syndrome and reagent kit of the biomarker.

### BACKGROUND

APS, also known as antiphospholipid syndrome, is a series of syndromes clinically manifested by recurrent arteriovenous thrombosis or habitual abortion and accompanied by antiphospholipid antibody (APA) positive persistently, which were first described by Harris EN in 1985. The basic pathological change is intravascular thrombosis, arteriovenous vascular thrombosis at all levels can cause corresponding symptoms, and small placental vascular thrombosis can cause abortion. Therefore, thrombosis is the most prominent clinical manifestation of APS and the main reason for acquired thrombosis currently recognized.

Symptoms of APS, in the skin, include livedo reticularis, skin ulcers, thrombophlebitis, acral necrosis, livedo reticularis cerebral thrombosis (Sneddon syndrome), Behcet's disease and malignant atrophic papulosis rash. In the nervous system, they can include migraine, chorea, local cerebral ischemia, lupus sclerosis, Jamaican myelopathy, and Guillain-Barré syndrome. Symptoms of visceral infarction and thrombosis can include cerebral infarction, cerebral venous thrombosis, myocardial infarction, renal infarction, pulmonary infarction, liver Budd-Chiari syndrome, aseptic bone necrosis, habitual abortion, retinal arteriovenous thrombosis and arterial occlusion of extremities.

It is necessary to provide biomarkers for determining the antiphospholipid syndrome for the judgment of APS.

### SUMMARY

The purpose of the present disclosure is to provide a biomarker for determining the antiphospholipid syndrome and its reagent kit.

The above object of the present disclosure is achieved by the following technical means.

In particular, the present disclosure describes applications of antibodies to MYO5A protein as a biomarker in terms of determining antiphospholipid syndrome.

Preferably, antibodies to the MYO5A protein include at least one of Ig IgM, or IgA.

Preferably, the above-mentioned applications of antibodies to MYO5A protein as a biomarker in terms of determining antiphospholipid syndrome are used to identify primary antiphospholipid syndrome.

Another preferred, the above-mentioned applications of antibodies to MYO5A protein as a biomarker in terms of determining antiphospholipid syndrome are used to identify secondary antiphospholipid syndrome.

Another preferred, the above-mentioned applications of antibodies to MYO5A protein as a biomarker in terms of determining antiphospholipid syndrome are used to identify primary antiphospholipid syndrome and secondary antiphospholipid syndrome simultaneously.

Preferably, the above-mentioned applications of antibodies to MYO5A protein as a biomarker in terms of determining antiphospholipid syndrome, wherein the sequence of MYO5A protein is,

```
Met Ala Ala Ser Glu Leu Tyr Thr Lys Phe Ala Arg Val Trp Ile Pro
1 5 10 15
Asp Pro Glu Glu Val Trp Lys Ser Ala Glu Leu Leu Lys Asp Tyr Lys
                        20 25 30
Pro Gly Asp Lys Val Leu Leu Leu His Leu Glu Glu Gly Lys Asp Leu
                35 40 45
Glu Tyr His Leu Asp Pro Lys Thr Lys Glu Leu Pro His Leu Arg Asn
        50 55 60
Pro Asp Ile Leu Val Gly Glu Asn Asp Leu Thr Ala Leu Ser Tyr Leu
65 70 75 80
His Glu Pro Ala Val Leu His Asn Leu Arg Val Arg Phe Ile Asp Ser
                                85 90 95
Lys Leu Ile Tyr Thr Tyr Cys Gly Ile Val Leu Val Ala Ile Asn Pro
                        100 105 110
Tyr Glu Gln Leu Pro Ile Tyr Gly Glu Asp Ile Ile Asn Ala Tyr Ser
                115 120 125
Gly Gln Asn Met Gly Asp Met Asp Pro His Ile Phe Ala Val Ala Glu
        130 135 140
Glu Ala Tyr Lys Gln Met Ala Arg Asp Glu Arg Asn Gln Ser Ile Ile
145 150 155 160
Val Ser Gly Glu Ser Gly Ala Gly Lys Thr Val Ser Ala Lys Tyr Ala
                                165 170 175
Met Arg Tyr Phe Ala Thr Val Ser Gly Ser Ala Ser Glu Ala Asn Val
                        180 185 190
Glu Glu Lys Val Leu Ala Ser Asn Pro Ile Met Glu Ser Ile Gly Asn
                195 200 205
Ala Lys Thr Thr Arg Asn Asp Asn Ser Ser Arg Phe Gly Lys Tyr Ile
        210 215 220
Glu Ile Gly Phe Asp Lys Arg Tyr Arg Ile Ile Gly Ala Asn Met Arg
225 230 235 240
Thr Tyr Leu Leu Glu Lys Ser Arg Val Val Phe Gln Ala Glu Glu Glu
                                245 250 255
Arg Asn Tyr His Ile Phe Tyr Gln Leu Cys Ala Ser Ala Lys Leu Pro
                        260 265 270
Glu Phe Lys Met Leu Arg Leu Gly Asn Ala Asp Asn Phe Asn Tyr Thr
                275 280 285
Lys Gln Gly Gly Ser Pro Val Ile Glu Gly Val Asp Asp Ala Lys Glu
        290 295 300
Met Ala His Thr Arg Gln Ala Cys Thr Leu Leu Gly Ile Ser Glu Ser
305 310 315 320
His Gln Met Gly Ile Phe Arg Ile Leu Ala Gly Ile Leu His Leu Gly
                                325 330 335
Asn Val Gly Phe Thr Ser Arg Asp Ala Asp Ser Cys Thr Ile Pro Pro
                        340 345 350
Lys His Glu Pro Leu Cys Ile Phe Cys Glu Leu Met Gly Val Asp Tyr
                355 360 365
Glu Glu Met Cys His Trp Leu Cys His Arg Lys Leu Ala Thr Ala Thr
        370 375 380
Glu Thr Tyr Ile Lys Pro Ile Ser Lys Leu Gln Ala Thr Asn Ala Arg
385 390 395 400
Asp Ala Leu Ala Lys His Ile Tyr Ala Lys Leu Phe Asn Trp Ile Val
                                405 410 415
Asp Asn Val Asn Gln Ala Leu His Ser Ala Val Lys Gln His Ser Phe
                        420 425 430
Ile Gly Val Leu Asp Ile Tyr Gly Phe Glu Thr Phe Glu Ile Asn Ser
                435 440 445
Phe Glu Gln Phe Cys Ile Asn Tyr Ala Asn Glu Lys Leu Gln Gln Gln
        450 455 460
Phe Asn Met His Val Phe Lys Leu Glu Gln Glu Glu Tyr Met Lys Glu
465 470 475 480
Gln Ile Pro Trp Thr Leu Ile Asp Phe Tyr Asp Asn Gln Pro Cys Ile
                                485 490 495
Asn Leu Ile Glu Ser Lys Leu Gly Ile Leu Asp Leu Leu Asp Glu Glu
                        500 505 510
Cys Lys Met Pro Lys Gly Thr Asp Asp Thr Trp Ala Gln Lys Leu Tyr
                515 520 525
Asn Thr His Leu Asn Lys Cys Ala Leu Phe Glu Lys Pro Arg Leu Ser
        530 535 540
Asn Lys Ala Phe Ile Ile Gln His Phe Ala Asp Lys Val Glu Tyr Gln
545 550 555 560
Cys Glu Gly Phe Leu Glu Lys Asn Lys Asp Thr Val Phe Glu Glu Gln
                                565 570 575
Ile Lys Val Leu Lys Ser Ser Lys Phe Lys Met Leu Pro Glu Leu Phe
                        580 585 590
Gln Asp Asp Glu Lys Ala Ile Ser Pro Thr Ser Ala Thr Ser Ser Gly
                595 600 605
Arg Thr Pro Leu Thr Arg Thr Pro Ala Lys Pro Thr Lys Gly Arg Pro
        610 615 620
Gly Gln Met Ala Lys Glu His Lys Lys Thr Val Gly His Gln Phe Arg
625 630 635 640
Asn Ser Leu His Leu Leu Met Glu Thr Leu Asn Ala Thr Thr Pro His
                                645 650 655
Tyr Val Arg Cys Ile Lys Pro Asn Asp Phe Lys Phe Pro Phe Thr Phe
                        660 665 670
Asp Glu Lys Arg Ala Val Gln Gln Leu Arg Ala Cys Gly Val Leu Glu
                675 680 685
Thr Ile Arg Ile Ser Ala Ala Gly Phe Pro Ser Arg Trp Thr Tyr Gln
        690 695 700
Glu Phe Phe Ser Arg Tyr Arg Val Leu Met Lys Gln Lys Asp Val Leu
705 710 715 720
Ser Asp Arg Lys Gln Thr Cys Lys Asn Val Leu Glu Lys Leu Ile Leu
                                725 730 735
Asp Lys Asp Lys Tyr Gln Phe Gly Lys Thr Lys Ile Phe Phe Arg Ala
                        740 745 750
Gly Gln Val Ala Tyr Leu Glu Lys Leu Arg Ala Asp Lys Leu Arg Ala
                755 760 765
Ala Cys Ile Arg Ile Gln Lys Thr Ile Arg Gly Trp Leu Leu Arg Lys
        770 775 780
Lys Tyr Leu Arg Met Arg Lys Ala Ala Ile Thr Met Gln Arg Tyr Val
785 790 795 800
Arg Gly Tyr Gln Ala Arg Cys Tyr Ala Lys Phe Leu Arg Arg Thr Lys
                                805 810 815
Ala Ala Thr Ile Ile Gln Lys Tyr Trp Arg Met Tyr Val Val Arg Arg
                        820 825 830
Arg Tyr Lys Ile Arg Arg Ala Ala Thr Ile Val Leu Gln Ser Tyr Leu
                835 840 845
Arg Gly Phe Leu Ala Arg Asn Arg Tyr Arg Lys Ile Leu Arg Glu His
        850 855 860
Lys Ala Val Ile Ile Gln Lys Arg Val Arg Gly Trp Leu Ala Arg Thr
865 870 875 880
His Tyr Lys Arg Ser Met His Ala Ile Ile Tyr Leu Gln Cys Cys Phe
                                885 890 895
Arg Arg Met Met Ala Lys Arg Glu Leu Lys Lys Leu Lys Ile Glu Ala
                        900 905 910
Arg Ser Val Glu Arg Tyr Lys Lys Leu His Ile Gly Met Glu Asn Lys
                915 920 925
Ile Met Gln Leu Gln Arg Lys Val Asp Glu Gln Asn Lys Asp Tyr Lys
        930 935 940
Cys Leu Val Glu Lys Leu Thr Asn Leu Glu Gly Ile Tyr Asn Ser Glu
945 950 955 960
Thr Glu Lys Leu Arg Ser Asp Leu Glu Arg Leu Gln Leu Ser Glu Glu
                                965 970 975
Glu Ala Lys Val Ala Thr Gly Arg Val Leu Ser Leu Gln Glu Glu Ile
                        980 985 990
Ala Lys Leu Arg Lys Asp Leu Glu Gln Thr Arg Ser Glu Lys Lys Cys
                995 1000 1005
Ile Glu Glu His Ala Asp Arg Tyr Lys Gln Glu Thr Glu Gln Leu
        1010 1015 1020
Val Ser Asn Leu Lys Glu Glu Asn Thr Leu Leu Lys Gln Glu Lys
        1025 1030 1035
Glu Ala Leu Asn His Arg Ile Val Gln Gln Ala Lys Glu Met Thr
        1040 1045 1050
Glu Thr Met Glu Lys Lys Leu Val Glu Glu Thr Lys Gln Leu Glu
        1055 1060 1065
Leu Asp Leu Asn Asp Glu Arg Leu Arg Tyr Gln Asn Leu Leu Asn
        1070 1075 1080
Glu Phe Ser Arg Leu Glu Glu Arg Tyr Asp Asp Leu Lys Glu Glu
        1085 1090 1095
Met Thr Leu Met Val His Val Pro Lys Pro Gly His Lys Arg Thr
        1100 1105 1110
Asp Ser Thr His Ser Ser Asn Glu Ser Glu Tyr Ile Phe Ser Ser
        1115 1120 1125
Glu Ile Ala Glu Met Glu Asp Ile Pro Ser Arg Thr Glu Glu Pro
        1130 1135 1140
Ser Glu Lys Lys Val Pro Leu Asp Met Ser Leu Phe Leu Lys Leu
        1145 1150 1155
Gln Lys Arg Val Thr Glu Leu Glu Gln Glu Lys Gln Val Met Gln
        1160 1165 1170
Asp Glu Leu Asp Arg Lys Glu Glu Gln Val Leu Arg Ser Lys Ala
        1175 1180 1185
Lys Glu Glu Glu Arg Pro Gln Ile Arg Gly Ala Glu Leu Glu Tyr
        1190 1195 1200
Glu Ser Leu Lys Arg Gln Glu Leu Glu Ser Glu Asn Lys Lys Leu
        1205 1210 1215
Lys Asn Glu Leu Asn Glu Leu Arg Lys Ala Leu Ser Glu Lys Ser
        1220 1225 1230
Ala Pro Glu Val Thr Ala Pro Gly Ala Pro Ala Tyr Arg Val Leu
        1235 1240 1245
Met Glu Gln Leu Thr Ser Val Ser Glu Glu Leu Asp Val Arg Lys
        1250 1255 1260
Glu Glu Val Leu Ile Leu Arg Ser Gln Leu Val Ser Gln Lys Glu
        1265 1270 1275
Ala Ile Gln Pro Lys Asp Asp Lys Asn Thr Met Thr Asp Ser Thr
        1280 1285 1290
Ile Leu Leu Glu Asp Val Gln Lys Met Lys Asp Lys Gly Glu Ile
        1295 1300 1305
Ala Gln Ala Tyr Ile Gly Leu Lys Glu Thr Asn Arg Ser Ser Ala
        1310 1315 1320
Leu Asp Tyr His Glu Leu Asn Glu Asp Gly Glu Leu Trp Leu Val
        1325 1330 1335
Tyr Glu Gly Leu Lys Gln Ala Asn Arg Leu Leu Glu Ser Gln Leu
        1340 1345 1350
Gln Ser Gln Lys Arg Ser His Glu Asn Glu Ala Glu Ala Leu Arg
        1355 1360 1365
Gly Glu Ile Gln Ser Leu Lys Glu Glu Asn Asn Arg Gln Gln Gln
        1370 1375 1380
Leu Leu Ala Gln Asn Leu Gln Leu Pro Pro Glu Ala Arg Ile Glu
        1385 1390 1395
Ala Ser Leu Gln His Glu Ile Thr Arg Leu Thr Asn Glu Asn Leu
        1400 1405 1410
Asp Leu Met Glu Gln Leu Glu Lys Gln Asp Lys Thr Val Arg Lys
        1415 1420 1425
Leu Lys Lys Gln Leu Lys Val Phe Ala Lys Lys Ile Gly Glu Leu
        1430 1435 1440
Glu Val Gly Gln Met Glu Asn Ile Ser Pro Gly Gln Ile Ile Asp
        1445 1450 1455
Glu Pro Ile Arg Pro Val Asn Ile Pro Arg Lys Glu Lys Asp Phe
        1460 1465 1470
Gln Gly Met Leu Glu Tyr Lys Lys Glu Asp Glu Gln Lys Leu Val
        1475 1480 1485
Lys Asn Leu Ile Leu Glu Leu Lys Pro Arg Gly Val Ala Val Asn
        1490 1495 1500
Leu Ile Pro Gly Leu Pro Ala Tyr Ile Leu Phe Met Cys Val Arg
        1505 1510 1515
His Ala Asp Tyr Leu Asn Asp Asp Gln Lys Val Arg Ser Leu Leu
        1520 1525 1530
Thr Ser Thr Ile Asn Ser Ile Lys Lys Val Leu Lys Lys Arg Gly
        1535 1540 1545
Asp Asp Phe Glu Thr Val Ser Phe Trp Leu Ser Asn Thr Cys Arg
        1550 1555 1560
Phe Leu His Cys Leu Lys Gln Tyr Ser Gly Glu Glu Gly Phe Met
        1565 1570 1575
Lys His Asn Thr Ser Arg Gln Asn Glu His Cys Leu Thr Asn Phe
        1580 1585 1590
Asp Leu Ala Glu Tyr Arg Gln Val Leu Ser Asp Leu Ala Ile Gln
        1595 1600 1605
Ile Tyr Gln Gln Leu Val Arg Val Leu Glu Asn Ile Leu Gln Pro
        1610 1615 1620
Met Ile Val Ser Gly Met Leu Glu His Glu Thr Ile Gln Gly Val
        1625 1630 1635
Ser Gly Val Lys Pro Thr Gly Leu Arg Lys Arg Thr Ser Ser Ile
        1640 1645 1650
Ala Asp Glu Gly Thr Tyr Thr Leu Asp Ser Ile Leu Arg Gln Leu
        1655 1660 1665
Asn Ser Phe His Ser Val Met Cys Gln His Gly Met Asp Pro Glu
        1670 1675 1680
Leu Ile Lys Gln Val Val Lys Gln Met Phe Tyr Ile Ile Gly Ala
        1685 1690 1695
Ile Thr Leu Asn Asn Leu Leu Leu Arg Lys Asp Met Cys Ser Trp
        1700 1705 1710
Ser Lys Gly Met Gln Ile Arg Tyr Asn Val Ser Gln Leu Glu Glu
        1715 1720 1725
Trp Leu Arg Asp Lys Asn Leu Met Asn Ser Gly Ala Lys Glu Thr
        1730 1735 1740
Leu Glu Pro Leu Ile Gln Ala Ala Gln Leu Leu Gln Val Lys Lys
        1745 1750 1755
Lys Thr Asp Asp Asp Ala Glu Ala Ile Cys Ser Met Cys Asn Ala
        1760 1765 1770
Leu Thr Thr Ala Gln Ile Val Lys Val Leu Asn Leu Tyr Thr Pro
        1775 1780 1785
Val Asn Glu Phe Glu Glu Arg Val Ser Val Ser Phe Ile Arg Thr
        1790 1795 1800
Ile Gln Met Arg Leu Arg Asp Arg Lys Asp Ser Pro Gln Leu Leu
        1805 1810 1815
Met Asp Ala Lys His Ile Phe Pro Val Thr Phe Pro Phe Asn Pro
        1820 1825 1830
Ser Ser Leu Ala Leu Glu Thr Ile Gln Ile Pro Ala Ser Leu Gly
        1835 1840 1845
Leu Gly Phe Ile Ser Arg Val
        1850 1855.
```

The present disclosure also provides a biomarker, which is an antibody produced by the MYO5A protein, determining whether there is antiphospholipid syndrome by detecting the presence of antibodies to MYO5A protein.

The present disclosure also provides a reagent kit, which is made of MYO5A protein, determining whether there is antiphospholipid syndrome by detecting the presence of antibodies to MYO5A protein.

Further, in the above reagent kit, the MYO5A protein or a partial sequence fragment thereof is selected as the core part, and the detection method is any one of ELISA, chemiluminescence or POCT.

Further, in the above reagent kit, the MYO5A protein is used as the core material, and the target molecule detected and identified by the reagent kit is the antibody produced by the MYO5A protein, including at least one of IgG, IgM or IgA. The samples detected include serum, plasma, or tissue fluid.

The present disclosure, applications of antibodies to MYO5A protein as a biomarker in terms of determining antiphospholipid syndrome, provides a new biomarker to identify antiphospholipid syndrome and a new application direction which is of great significance for the identification of antiphospholipid syndrome.

### BRIEF DESCRIPTION OF THE DRAWINGS

The present disclosure is further described using the drawings, but the contents in the drawings do not constitute any limitation to the present disclosure.

FIG. 1 is a graph showing the different results of antibodies to MYO5A protein in the APS group and the disease control group.

FIG. 2 is a schematic diagram of the results of the significant difference between the antibodies to MYO5A protein in the APS group and in the healthy control group.

FIG. 3 is a schematic diagram of the difference between the antibodies to MYO5A protein in the primary APS and in the secondary APS group.

### DESCRIPTION OF THE EMBODIMENTS

The present disclosure is further described in conjunction with the following embodiments.

### Embodiment 1

Applications of antibodies to MYO5A protein as a biomarker in terms of determining antiphospholipid syndrome, wherein antibodies to the MYO5A protein include at least one of IgG, IgM, or IgA.

Applications of antibodies to MYO5A protein as a biomarker in terms of determining antiphospholipid syndrome can be used to identify primary antiphospholipid syndrome, secondary antiphospholipid syndrome or to identify primary antiphospholipid syndrome and secondary antiphospholipid syndrome simultaneously.

The sequence of MYO5A protein is,

```
Met Ala Ala Ser Glu Leu Tyr Thr Lys Phe Ala Arg Val Trp Ile Pro
1 5 10 15
Asp Pro Glu Glu Val Trp Lys Ser Ala Glu Leu Leu Lys Asp Tyr Lys
                        20 25 30
Pro Gly Asp Lys Val Leu Leu Leu His Leu Glu Glu Gly Lys Asp Leu
                35 40 45
Glu Tyr His Leu Asp Pro Lys Thr Lys Glu Leu Pro His Leu Arg Asn
        50 55 60
Pro Asp Ile Leu Val Gly Glu Asn Asp Leu Thr Ala Leu Ser Tyr Leu
65 70 75 80
His Glu Pro Ala Val Leu His Asn Leu Arg Val Arg Phe Ile Asp Ser
                                85 90 95
Lys Leu Ile Tyr Thr Tyr Cys Gly Ile Val Leu Val Ala Ile Asn Pro
                        100 105 110
Tyr Glu Gln Leu Pro Ile Tyr Gly Glu Asp Ile Ile Asn Ala Tyr Ser
                115 120 125
Gly Gln Asn Met Gly Asp Met Asp Pro His Ile Phe Ala Val Ala Glu
        130 135 140
Glu Ala Tyr Lys Gln Met Ala Arg Asp Glu Arg Asn Gln Ser Ile Ile
145 150 155 160
Val Ser Gly Glu Ser Gly Ala Gly Lys Thr Val Ser Ala Lys Tyr Ala
                                165 170 175
Met Arg Tyr Phe Ala Thr Val Ser Gly Ser Ala Ser Glu Ala Asn Val
                        180 185 190
Glu Glu Lys Val Leu Ala Ser Asn Pro Ile Met Glu Ser Ile Gly Asn
                195 200 205
Ala Lys Thr Thr Arg Asn Asp Asn Ser Ser Arg Phe Gly Lys Tyr Ile
        210 215 220
Glu Ile Gly Phe Asp Lys Arg Tyr Arg Ile Ile Gly Ala Asn Met Arg
225 230 235 240
Thr Tyr Leu Leu Glu Lys Ser Arg Val Val Phe Gln Ala Glu Glu Glu
                                245 250 255
Arg Asn Tyr His Ile Phe Tyr Gln Leu Cys Ala Ser Ala Lys Leu Pro
                        260 265 270
Glu Phe Lys Met Leu Arg Leu Gly Asn Ala Asp Asn Phe Asn Tyr Thr
                275 280 285
Lys Gln Gly Gly Ser Pro Val Ile Glu Gly Val Asp Asp Ala Lys Glu
        290 295 300
Met Ala His Thr Arg Gln Ala Cys Thr Leu Leu Gly Ile Ser Glu Ser
305 310 315 320
His Gln Met Gly Ile Phe Arg Ile Leu Ala Gly Ile Leu His Leu Gly
                                325 330 335
Asn Val Gly Phe Thr Ser Arg Asp Ala Asp Ser Cys Thr Ile Pro Pro
                        340 345 350
Lys His Glu Pro Leu Cys Ile Phe Cys Glu Leu Met Gly Val Asp Tyr
                355 360 365
Glu Glu Met Cys His Trp Leu Cys His Arg Lys Leu Ala Thr Ala Thr
        370 375 380
Glu Thr Tyr Ile Lys Pro Ile Ser Lys Leu Gln Ala Thr Asn Ala Arg
385 390 395 400
Asp Ala Leu Ala Lys His Ile Tyr Ala Lys Leu Phe Asn Trp Ile Val
                                405 410 415
Asp Asn Val Asn Gln Ala Leu His Ser Ala Val Lys Gln His Ser Phe
                        420 425 430
Ile Gly Val Leu Asp Ile Tyr Gly Phe Glu Thr Phe Glu Ile Asn Ser
                435 440 445
Phe Glu Gln Phe Cys Ile Asn Tyr Ala Asn Glu Lys Leu Gln Gln Gln
        450 455 460
Phe Asn Met His Val Phe Lys Leu Glu Gln Glu Glu Tyr Met Lys Glu
465 470 475 480
Gln Ile Pro Trp Thr Leu Ile Asp Phe Tyr Asp Asn Gln Pro Cys Ile
                                485 490 495
Asn Leu Ile Glu Ser Lys Leu Gly Ile Leu Asp Leu Leu Asp Glu Glu
                        500 505 510
Cys Lys Met Pro Lys Gly Thr Asp Asp Thr Trp Ala Gln Lys Leu Tyr
                515 520 525
Asn Thr His Leu Asn Lys Cys Ala Leu Phe Glu Lys Pro Arg Leu Ser
        530 535 540
Asn Lys Ala Phe Ile Ile Gln His Phe Ala Asp Lys Val Glu Tyr Gln
545 550 555 560
Cys Glu Gly Phe Leu Glu Lys Asn Lys Asp Thr Val Phe Glu Glu Gln
                                565 570 575
Ile Lys Val Leu Lys Ser Ser Lys Phe Lys Met Leu Pro Glu Leu Phe
                        580 585 590
Gln Asp Asp Glu Lys Ala Ile Ser Pro Thr Ser Ala Thr Ser Ser Gly
                595 600 605
Arg Thr Pro Leu Thr Arg Thr Pro Ala Lys Pro Thr Lys Gly Arg Pro
        610 615 620
Gly Gln Met Ala Lys Glu His Lys Lys Thr Val Gly His Gln Phe Arg
625 630 635 640
Asn Ser Leu His Leu Leu Met Glu Thr Leu Asn Ala Thr Thr Pro His
                                645 650 655
Tyr Val Arg Cys Ile Lys Pro Asn Asp Phe Lys Phe Pro Phe Thr Phe
                        660 665 670
Asp Glu Lys Arg Ala Val Gln Gln Leu Arg Ala Cys Gly Val Leu Glu
                675 680 685
Thr Ile Arg Ile Ser Ala Ala Gly Phe Pro Ser Arg Trp Thr Tyr Gln
        690 695 700
Glu Phe Phe Ser Arg Tyr Arg Val Leu Met Lys Gln Lys Asp Val Leu
705 710 715 720
Ser Asp Arg Lys Gln Thr Cys Lys Asn Val Leu Glu Lys Leu Ile Leu
                                725 730 735
Asp Lys Asp Lys Tyr Gln Phe Gly Lys Thr Lys Ile Phe Phe Arg Ala
                        740 745 750
Gly Gln Val Ala Tyr Leu Glu Lys Leu Arg Ala Asp Lys Leu Arg Ala
                755 760 765
Ala Cys Ile Arg Ile Gln Lys Thr Ile Arg Gly Trp Leu Leu Arg Lys
        770 775 780
Lys Tyr Leu Arg Met Arg Lys Ala Ala Ile Thr Met Gln Arg Tyr Val
785 790 795 800
Arg Gly Tyr Gln Ala Arg Cys Tyr Ala Lys Phe Leu Arg Arg Thr Lys
                                805 810 815
Ala Ala Thr Ile Ile Gln Lys Tyr Trp Arg Met Tyr Val Val Arg Arg
                        820 825 830
Arg Tyr Lys Ile Arg Arg Ala Ala Thr Ile Val Leu Gln Ser Tyr Leu
                835 840 845
Arg Gly Phe Leu Ala Arg Asn Arg Tyr Arg Lys Ile Leu Arg Glu His
        850 855 860
Lys Ala Val Ile Ile Gln Lys Arg Val Arg Gly Trp Leu Ala Arg Thr
865 870 875 880
His Tyr Lys Arg Ser Met His Ala Ile Ile Tyr Leu Gln Cys Cys Phe
                                885 890 895
Arg Arg Met Met Ala Lys Arg Glu Leu Lys Lys Leu Lys Ile Glu Ala
                        900 905 910
Arg Ser Val Glu Arg Tyr Lys Lys Leu His Ile Gly Met Glu Asn Lys
                915 920 925
Ile Met Gln Leu Gln Arg Lys Val Asp Glu Gln Asn Lys Asp Tyr Lys
        930 935 940
Cys Leu Val Glu Lys Leu Thr Asn Leu Glu Gly Ile Tyr Asn Ser Glu
945 950 955 960
Thr Glu Lys Leu Arg Ser Asp Leu Glu Arg Leu Gln Leu Ser Glu Glu
                                965 970 975
Glu Ala Lys Val Ala Thr Gly Arg Val Leu Ser Leu Gln Glu Glu Ile
                        980 985 990
Ala Lys Leu Arg Lys Asp Leu Glu Gln Thr Arg Ser Glu Lys Lys Cys
                995 1000 1005
Ile Glu Glu His Ala Asp Arg Tyr Lys Gln Glu Thr Glu Gln Leu
        1010 1015 1020
Val Ser Asn Leu Lys Glu Glu Asn Thr Leu Leu Lys Gln Glu Lys
        1025 1030 1035
Glu Ala Leu Asn His Arg Ile Val Gln Gln Ala Lys Glu Met Thr
        1040 1045 1050
Glu Thr Met Glu Lys Lys Leu Val Glu Glu Thr Lys Gln Leu Glu
        1055 1060 1065
Leu Asp Leu Asn Asp Glu Arg Leu Arg Tyr Gln Asn Leu Leu Asn
        1070 1075 1080
Glu Phe Ser Arg Leu Glu Glu Arg Tyr Asp Asp Leu Lys Glu Glu
        1085 1090 1095
Met Thr Leu Met Val His Val Pro Lys Pro Gly His Lys Arg Thr
        1100 1105 1110
Asp Ser Thr His Ser Ser Asn Glu Ser Glu Tyr Ile Phe Ser Ser
        1115 1120 1125
Glu Ile Ala Glu Met Glu Asp Ile Pro Ser Arg Thr Glu Glu Pro
        1130 1135 1140
Ser Glu Lys Lys Val Pro Leu Asp Met Ser Leu Phe Leu Lys Leu
        1145 1150 1155
Gln Lys Arg Val Thr Glu Leu Glu Gln Glu Lys Gln Val Met Gln
        1160 1165 1170
Asp Glu Leu Asp Arg Lys Glu Glu Gln Val Leu Arg Ser Lys Ala
        1175 1180 1185
Lys Glu Glu Glu Arg Pro Gln Ile Arg Gly Ala Glu Leu Glu Tyr
        1190 1195 1200
Glu Ser Leu Lys Arg Gln Glu Leu Glu Ser Glu Asn Lys Lys Leu
        1205 1210 1215
Lys Asn Glu Leu Asn Glu Leu Arg Lys Ala Leu Ser Glu Lys Ser
        1220 1225 1230
Ala Pro Glu Val Thr Ala Pro Gly Ala Pro Ala Tyr Arg Val Leu
        1235 1240 1245
Met Glu Gln Leu Thr Ser Val Ser Glu Glu Leu Asp Val Arg Lys
        1250 1255 1260
Glu Glu Val Leu Ile Leu Arg Ser Gln Leu Val Ser Gln Lys Glu
        1265 1270 1275
Ala Ile Gln Pro Lys Asp Asp Lys Asn Thr Met Thr Asp Ser Thr
        1280 1285 1290
Ile Leu Leu Glu Asp Val Gln Lys Met Lys Asp Lys Gly Glu Ile
        1295 1300 1305
Ala Gln Ala Tyr Ile Gly Leu Lys Glu Thr Asn Arg Ser Ser Ala
        1310 1315 1320
Leu Asp Tyr His Glu Leu Asn Glu Asp Gly Glu Leu Trp Leu Val
        1325 1330 1335
Tyr Glu Gly Leu Lys Gln Ala Asn Arg Leu Leu Glu Ser Gln Leu
        1340 1345 1350
Gln Ser Gln Lys Arg Ser His Glu Asn Glu Ala Glu Ala Leu Arg
        1355 1360 1365
Gly Glu Ile Gln Ser Leu Lys Glu Glu Asn Asn Arg Gln Gln Gln
        1370 1375 1380
Leu Leu Ala Gln Asn Leu Gln Leu Pro Pro Glu Ala Arg Ile Glu
        1385 1390 1395
Ala Ser Leu Gln His Glu Ile Thr Arg Leu Thr Asn Glu Asn Leu
        1400 1405 1410
Asp Leu Met Glu Gln Leu Glu Lys Gln Asp Lys Thr Val Arg Lys
        1415 1420 1425
Leu Lys Lys Gln Leu Lys Val Phe Ala Lys Lys Ile Gly Glu Leu
        1430 1435 1440
Glu Val Gly Gln Met Glu Asn Ile Ser Pro Gly Gln Ile Ile Asp
        1445 1450 1455
Glu Pro Ile Arg Pro Val Asn Ile Pro Arg Lys Glu Lys Asp Phe
        1460 1465 1470
Gln Gly Met Leu Glu Tyr Lys Lys Glu Asp Glu Gln Lys Leu Val
        1475 1480 1485
Lys Asn Leu Ile Leu Glu Leu Lys Pro Arg Gly Val Ala Val Asn
        1490 1495 1500
Leu Ile Pro Gly Leu Pro Ala Tyr Ile Leu Phe Met Cys Val Arg
        1505 1510 1515
His Ala Asp Tyr Leu Asn Asp Asp Gln Lys Val Arg Ser Leu Leu
        1520 1525 1530
Thr Ser Thr Ile Asn Ser Ile Lys Lys Val Leu Lys Lys Arg Gly
        1535 1540 1545
Asp Asp Phe Glu Thr Val Ser Phe Trp Leu Ser Asn Thr Cys Arg
        1550 1555 1560
Phe Leu His Cys Leu Lys Gln Tyr Ser Gly Glu Glu Gly Phe Met
        1565 1570 1575
Lys His Asn Thr Ser Arg Gln Asn Glu His Cys Leu Thr Asn Phe
        1580 1585 1590
Asp Leu Ala Glu Tyr Arg Gln Val Leu Ser Asp Leu Ala Ile Gln
        1595 1600 1605
Ile Tyr Gln Gln Leu Val Arg Val Leu Glu Asn Ile Leu Gln Pro
        1610 1615 1620
Met Ile Val Ser Gly Met Leu Glu His Glu Thr Ile Gln Gly Val
        1625 1630 1635
Ser Gly Val Lys Pro Thr Gly Leu Arg Lys Arg Thr Ser Ser Ile
        1640 1645 1650
Ala Asp Glu Gly Thr Tyr Thr Leu Asp Ser Ile Leu Arg Gln Leu
        1655 1660 1665
Asn Ser Phe His Ser Val Met Cys Gln His Gly Met Asp Pro Glu
        1670 1675 1680
Leu Ile Lys Gln Val Val Lys Gln Met Phe Tyr Ile Ile Gly Ala
        1685 1690 1695
Ile Thr Leu Asn Asn Leu Leu Leu Arg Lys Asp Met Cys Ser Trp
        1700 1705 1710
Ser Lys Gly Met Gln Ile Arg Tyr Asn Val Ser Gln Leu Glu Glu
        1715 1720 1725
Trp Leu Arg Asp Lys Asn Leu Met Asn Ser Gly Ala Lys Glu Thr
        1730 1735 1740
Leu Glu Pro Leu Ile Gln Ala Ala Gln Leu Leu Gln Val Lys Lys
        1745 1750 1755
Lys Thr Asp Asp Asp Ala Glu Ala Ile Cys Ser Met Cys Asn Ala
        1760 1765 1770
Leu Thr Thr Ala Gln Ile Val Lys Val Leu Asn Leu Tyr Thr Pro
        1775 1780 1785
Val Asn Glu Phe Glu Glu Arg Val Ser Val Ser Phe Ile Arg Thr
        1790 1795 1800
Ile Gln Met Arg Leu Arg Asp Arg Lys Asp Ser Pro Gln Leu Leu
        1805 1810 1815
Met Asp Ala Lys His Ile Phe Pro Val Thr Phe Pro Phe Asn Pro
        1820 1825 1830
Ser Ser Leu Ala Leu Glu Thr Ile Gln Ile Pro Ala Ser Leu Gly
        1835 1840 1845
Leu Gly Phe Ile Ser Arg Val
        1850 1855.
```

The above results are verified through experiments:

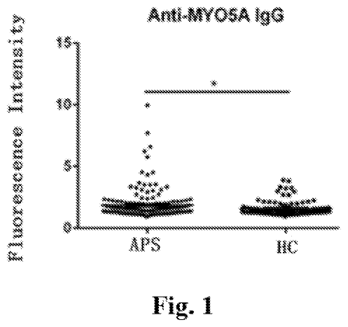

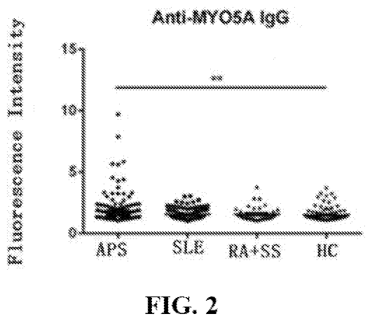

There is no significant difference between the antibodies to MYO5A protein in the APS group and in the disease control group, as shown in FIG. 1. The antibodies to MYO5A protein only show a significant difference between the APS group and the healthy control group, as shown in FIG. 2.

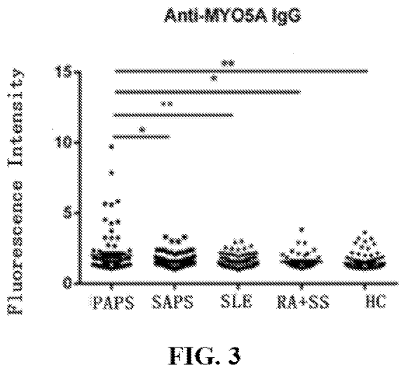

The APS group is divided into primary APS and secondary APS group in order to find differential protein antibodies between the subgroups, in which the results are shown in FIG. 3. The experiments find that the expression of antibodies to MYO5A protein exists a significant difference between the primary APS and secondary APS group, and also exists among the primary APS group, other disease control group and the healthy control group.

The present disclosure, applications of antibodies to MYO5A protein as a biomarker in terms of determining antiphospholipid syndrome, provides a new biomarker to identify antiphospholipid syndrome and a new application direction which is of great significance for the identification of antiphospholipid syndrome.

### Embodiment 2

The present embodiment provides a biomarker, which is an antibody produced by the MYO5A protein, determining whether there is antiphospholipid syndrome by detecting the presence of antibodies to MYO5A protein.

### Embodiment 3

The present embodiment provides a reagent kit, which is made of MYO5A protein, determining whether there is antiphospholipid syndrome by detecting the presence of antibodies to MYO5A protein.

Specifically, in the reagent kit, the MYO5A protein or a partial sequence fragment thereof is selected as the core part, and the detection method is any one of ELISA, chemiluminescence or POCT.

In the reagent kit, the MYO5A protein is used as the core material, and the target molecule detected and identified by the reagent kit is the antibody produced by the MYO5A protein, including at least one of IgG, IgM or IgA. The samples detected include serum, plasma, or tissue fluid.

The present disclosure, applications of antibodies to MYO5A protein as a biomarker in terms of determining antiphospholipid syndrome, provides a new biomarker to identify antiphospholipid syndrome and a new reagent kit, which is of great significance for the identification of antiphospholipid syndrome.

Finally, it should be noted that the above embodiments are merely used for describing the technical scheme of the present disclosure, but not intended to limit the protection scope of the present disclosure. Although the present disclosure is described in detail according to preferred embodiments, those of ordinary skills in the art should understand that the technical schemes of the present disclosure may be modified or equivalently substituted without departing from the spirit and protection scope of the present disclosure.

What is claimed is:

1. Applications of antibodies to MYO5A protein as a biomarker in terms of determining antiphospholipid syndrome.

2. Applications of antibodies to MYO5A protein as a biomarker in terms of determining antiphospholipid syndrome according to claim 1, wherein antibodies to the MYO5A protein include at least one of IgG, IgM or IgA.

3. Applications of antibodies to MYO5A protein as a biomarker in terms of determining antiphospholipid syndrome according to claim 2, are used to identify primary antiphospholipid syndrome.

4. Applications of antibodies to MYO5A protein as a biomarker in terms of determining antiphospholipid syndrome according to claim 2, are used to identify secondary antiphospholipid syndrome.

5. Applications of antibodies to MYO5A protein as a biomarker in terms of determining antiphospholipid syndrome according to claim 2, are used to identify primary antiphospholipid syndrome and secondary antiphospholipid syndrome simultaneously.

6. Applications of antibodies to MYO5A protein as a biomarker in terms of determining antiphospholipid syndrome according to claim 1, wherein the sequence of MYO5A protein is,
```
Met Ala Ala Ser Glu Leu Tyr Thr Lys Phe Ala Arg Val Trp Ile Pro
1 5 10 15
Asp Pro Glu Glu Val Trp Lys Ser Ala Glu Leu Leu Lys Asp Tyr Lys
                        20 25 30
Pro Gly Asp Lys Val Leu Leu Leu His Leu Glu Glu Gly Lys Asp Leu
                35 40 45
Glu Tyr His Leu Asp Pro Lys Thr Lys Glu Leu Pro His Leu Arg Asn
        50 55 60
Pro Asp Ile Leu Val Gly Glu Asn Asp Leu Thr Ala Leu Ser Tyr Leu
65 70 75 80
His Glu Pro Ala Val Leu His Asn Leu Arg Val Arg Phe Ile Asp Ser
                                85 90 95
Lys Leu Ile Tyr Thr Tyr Cys Gly Ile Val Leu Val Ala Ile Asn Pro
                        100 105 110
Tyr Glu Gln Leu Pro Ile Tyr Gly Glu Asp Ile Ile Asn Ala Tyr Ser
                115 120 125
Gly Gln Asn Met Gly Asp Met Asp Pro His Ile Phe Ala Val Ala Glu
        130 135 140
Glu Ala Tyr Lys Gln Met Ala Arg Asp Glu Arg Asn Gln Ser Ile Ile
145 150 155 160
Val Ser Gly Glu Ser Gly Ala Gly Lys Thr Val Ser Ala Lys Tyr Ala
                                165 170 175
Met Arg Tyr Phe Ala Thr Val Ser Gly Ser Ala Ser Glu Ala Asn Val
                        180 185 190
Glu Glu Lys Val Leu Ala Ser Asn Pro Ile Met Glu Ser Ile Gly Asn
                195 200 205
Ala Lys Thr Thr Arg Asn Asp Asn Ser Ser Arg Phe Gly Lys Tyr Ile
        210 215 220
Glu Ile Gly Phe Asp Lys Arg Tyr Arg Ile Ile Gly Ala Asn Met Arg
225 230 235 240
Thr Tyr Leu Leu Glu Lys Ser Arg Val Val Phe Gln Ala Glu Glu Glu
                                245 250 255
Arg Asn Tyr His Ile Phe Tyr Gln Leu Cys Ala Ser Ala Lys Leu Pro
                        260 265 270
Glu Phe Lys Met Leu Arg Leu Gly Asn Ala Asp Asn Phe Asn Tyr Thr
                275 280 285
Lys Gln Gly Gly Ser Pro Val Ile Glu Gly Val Asp Asp Ala Lys Glu
        290 295 300
Met Ala His Thr Arg Gln Ala Cys Thr Leu Leu Gly Ile Ser Glu Ser
305 310 315 320
His Gln Met Gly Ile Phe Arg Ile Leu Ala Gly Ile Leu His Leu Gly
                                325 330 335
Asn Val Gly Phe Thr Ser Arg Asp Ala Asp Ser Cys Thr Ile Pro Pro
                        340 345 350
Lys His Glu Pro Leu Cys Ile Phe Cys Glu Leu Met Gly Val Asp Tyr
                355 360 365
Glu Glu Met Cys His Trp Leu Cys His Arg Lys Leu Ala Thr Ala Thr
        370 375 380
Glu Thr Tyr Ile Lys Pro Ile Ser Lys Leu Gln Ala Thr Asn Ala Arg
385 390 395 400
Asp Ala Leu Ala Lys His Ile Tyr Ala Lys Leu Phe Asn Trp Ile Val
                                405 410 415
Asp Asn Val Asn Gln Ala Leu His Ser Ala Val Lys Gln His Ser Phe
                        420 425 430
Ile Gly Val Leu Asp Ile Tyr Gly Phe Glu Thr Phe Glu Ile Asn Ser
                435 440 445
Phe Glu Gln Phe Cys Ile Asn Tyr Ala Asn Glu Lys Leu Gln Gln Gln
        450 455 460
Phe Asn Met His Val Phe Lys Leu Glu Gln Glu Glu Tyr Met Lys Glu
465 470 475 480
Gln Ile Pro Trp Thr Leu Ile Asp Phe Tyr Asp Asn Gln Pro Cys Ile
                                485 490 495
Asn Leu Ile Glu Ser Lys Leu Gly Ile Leu Asp Leu Leu Asp Glu Glu
                        500 505 510
Cys Lys Met Pro Lys Gly Thr Asp Asp Thr Trp Ala Gln Lys Leu Tyr
                515 520 525
Asn Thr His Leu Asn Lys Cys Ala Leu Phe Glu Lys Pro Arg Leu Ser
        530 535 540
Asn Lys Ala Phe Ile Ile Gln His Phe Ala Asp Lys Val Glu Tyr Gln
545 550 555 560
Cys Glu Gly Phe Leu Glu Lys Asn Lys Asp Thr Val Phe Glu Glu Gln
                                565 570 575
Ile Lys Val Leu Lys Ser Ser Lys Phe Lys Met Leu Pro Glu Leu Phe
                        580 585 590
Gln Asp Asp Glu Lys Ala Ile Ser Pro Thr Ser Ala Thr Ser Ser Gly
                595 600 605
Arg Thr Pro Leu Thr Arg Thr Pro Ala Lys Pro Thr Lys Gly Arg Pro
        610 615 620
Gly Gln Met Ala Lys Glu His Lys Lys Thr Val Gly His Gln Phe Arg
625 630 635 640
Asn Ser Leu His Leu Leu Met Glu Thr Leu Asn Ala Thr Thr Pro His
                                645 650 655
Tyr Val Arg Cys Ile Lys Pro Asn Asp Phe Lys Phe Pro Phe Thr Phe
                        660 665 670
Asp Glu Lys Arg Ala Val Gln Gln Leu Arg Ala Cys Gly Val Leu Glu
                675 680 685
Thr Ile Arg Ile Ser Ala Ala Gly Phe Pro Ser Arg Trp Thr Tyr Gln
        690 695 700
Glu Phe Phe Ser Arg Tyr Arg Val Leu Met Lys Gln Lys Asp Val Leu
705 710 715 720
Ser Asp Arg Lys Gln Thr Cys Lys Asn Val Leu Glu Lys Leu Ile Leu
                                725 730 735
Asp Lys Asp Lys Tyr Gln Phe Gly Lys Thr Lys Ile Phe Phe Arg Ala
                        740 745 750
Gly Gln Val Ala Tyr Leu Glu Lys Leu Arg Ala Asp Lys Leu Arg Ala
                755 760 765
Ala Cys Ile Arg Ile Gln Lys Thr Ile Arg Gly Trp Leu Leu Arg Lys
        770 775 780
Lys Tyr Leu Arg Met Arg Lys Ala Ala Ile Thr Met Gln Arg Tyr Val
785 790 795 800
Arg Gly Tyr Gln Ala Arg Cys Tyr Ala Lys Phe Leu Arg Arg Thr Lys
                                805 810 815
Ala Ala Thr Ile Ile Gln Lys Tyr Trp Arg Met Tyr Val Val Arg Arg
                        820 825 830
Arg Tyr Lys Ile Arg Arg Ala Ala Thr Ile Val Leu Gln Ser Tyr Leu
                835 840 845
Arg Gly Phe Leu Ala Arg Asn Arg Tyr Arg Lys Ile Leu Arg Glu His
        850 855 860
Lys Ala Val Ile Ile Gln Lys Arg Val Arg Gly Trp Leu Ala Arg Thr
865 870 875 880
His Tyr Lys Arg Ser Met His Ala Ile Ile Tyr Leu Gln Cys Cys Phe
                                885 890 895
Arg Arg Met Met Ala Lys Arg Glu Leu Lys Lys Leu Lys Ile Glu Ala
                        900 905 910
Arg Ser Val Glu Arg Tyr Lys Lys Leu His Ile Gly Met Glu Asn Lys
                915 920 925
Ile Met Gln Leu Gln Arg Lys Val Asp Glu Gln Asn Lys Asp Tyr Lys
        930 935 940
Cys Leu Val Glu Lys Leu Thr Asn Leu Glu Gly Ile Tyr Asn Ser Glu
945 950 955 960
Thr Glu Lys Leu Arg Ser Asp Leu Glu Arg Leu Gln Leu Ser Glu Glu
                                965 970 975
Glu Ala Lys Val Ala Thr Gly Arg Val Leu Ser Leu Gln Glu Glu Ile
                        980 985 990
Ala Lys Leu Arg Lys Asp Leu Glu Gln Thr Arg Ser Glu Lys Lys Cys
                995 1000 1005
Ile Glu Glu His Ala Asp Arg Tyr Lys Gln Glu Thr Glu Gln Leu
        1010 1015 1020
Val Ser Asn Leu Lys Glu Glu Asn Thr Leu Leu Lys Gln Glu Lys
        1025 1030 1035
Glu Ala Leu Asn His Arg Ile Val Gln Gln Ala Lys Glu Met Thr
        1040 1045 1050
Glu Thr Met Glu Lys Lys Leu Val Glu Glu Thr Lys Gln Leu Glu
        1055 1060 1065
Leu Asp Leu Asn Asp Glu Arg Leu Arg Tyr Gln Asn Leu Leu Asn
        1070 1075 1080
Glu Phe Ser Arg Leu Glu Glu Arg Tyr Asp Asp Leu Lys Glu Glu
        1085 1090 1095
Met Thr Leu Met Val His Val Pro Lys Pro Gly His Lys Arg Thr
        1100 1105 1110
Asp Ser Thr His Ser Ser Asn Glu Ser Glu Tyr Ile Phe Ser Ser
        1115 1120 1125
Glu Ile Ala Glu Met Glu Asp Ile Pro Ser Arg Thr Glu Glu Pro
        1130 1135 1140
Ser Glu Lys Lys Val Pro Leu Asp Met Ser Leu Phe Leu Lys Leu
        1145 1150 1155
Gln Lys Arg Val Thr Glu Leu Glu Gln Glu Lys Gln Val Met Gln
        1160 1165 1170
Asp Glu Leu Asp Arg Lys Glu Glu Gln Val Leu Arg Ser Lys Ala
        1175 1180 1185
Lys Glu Glu Glu Arg Pro Gln Ile Arg Gly Ala Glu Leu Glu Tyr
        1190 1195 1200
Glu Ser Leu Lys Arg Gln Glu Leu Glu Ser Glu Asn Lys Lys Leu
        1205 1210 1215
Lys Asn Glu Leu Asn Glu Leu Arg Lys Ala Leu Ser Glu Lys Ser
        1220 1225 1230
Ala Pro Glu Val Thr Ala Pro Gly Ala Pro Ala Tyr Arg Val Leu
        1235 1240 1245
Met Glu Gln Leu Thr Ser Val Ser Glu Glu Leu Asp Val Arg Lys
        1250 1255 1260
Glu Glu Val Leu Ile Leu Arg Ser Gln Leu Val Ser Gln Lys Glu
        1265 1270 1275
Ala Ile Gln Pro Lys Asp Asp Lys Asn Thr Met Thr Asp Ser Thr
        1280 1285 1290
Ile Leu Leu Glu Asp Val Gln Lys Met Lys Asp Lys Gly Glu Ile
        1295 1300 1305
Ala Gln Ala Tyr Ile Gly Leu Lys Glu Thr Asn Arg Ser Ser Ala
        1310 1315 1320
Leu Asp Tyr His Glu Leu Asn Glu Asp Gly Glu Leu Trp Leu Val
        1325 1330 1335
Tyr Glu Gly Leu Lys Gln Ala Asn Arg Leu Leu Glu Ser Gln Leu
        1340 1345 1350
Gln Ser Gln Lys Arg Ser His Glu Asn Glu Ala Glu Ala Leu Arg
        1355 1360 1365
Gly Glu Ile Gln Ser Leu Lys Glu Glu Asn Asn Arg Gln Gln Gln
        1370 1375 1380
Leu Leu Ala Gln Asn Leu Gln Leu Pro Pro Glu Ala Arg Ile Glu
        1385 1390 1395
Ala Ser Leu Gln His Glu Ile Thr Arg Leu Thr Asn Glu Asn Leu
        1400 1405 1410
Asp Leu Met Glu Gln Leu Glu Lys Gln Asp Lys Thr Val Arg Lys
        1415 1420 1425
Leu Lys Lys Gln Leu Lys Val Phe Ala Lys Lys Ile Gly Glu Leu
        1430 1435 1440
Glu Val Gly Gln Met Glu Asn Ile Ser Pro Gly Gln Ile Ile Asp
        1445 1450 1455
Glu Pro Ile Arg Pro Val Asn Ile Pro Arg Lys Glu Lys Asp Phe
        1460 1465 1470
Gln Gly Met Leu Glu Tyr Lys Lys Glu Asp Glu Gln Lys Leu Val
        1475 1480 1485
Lys Asn Leu Ile Leu Glu Leu Lys Pro Arg Gly Val Ala Val Asn
        1490 1495 1500
Leu Ile Pro Gly Leu Pro Ala Tyr Ile Leu Phe Met Cys Val Arg
        1505 1510 1515
His Ala Asp Tyr Leu Asn Asp Asp Gln Lys Val Arg Ser Leu Leu
        1520 1525 1530
Thr Ser Thr Ile Asn Ser Ile Lys Lys Val Leu Lys Lys Arg Gly
        1535 1540 1545
Asp Asp Phe Glu Thr Val Ser Phe Trp Leu Ser Asn Thr Cys Arg
        1550 1555 1560
Phe Leu His Cys Leu Lys Gln Tyr Ser Gly Glu Glu Gly Phe Met
        1565 1570 1575
Lys His Asn Thr Ser Arg Gln Asn Glu His Cys Leu Thr Asn Phe
        1580 1585 1590
Asp Leu Ala Glu Tyr Arg Gln Val Leu Ser Asp Leu Ala Ile Gln
        1595 1600 1605
Ile Tyr Gln Gln Leu Val Arg Val Leu Glu Asn Ile Leu Gln Pro
        1610 1615 1620
Met Ile Val Ser Gly Met Leu Glu His Glu Thr Ile Gln Gly Val
        1625 1630 1635
Ser Gly Val Lys Pro Thr Gly Leu Arg Lys Arg Thr Ser Ser Ile
        1640 1645 1650
Ala Asp Glu Gly Thr Tyr Thr Leu Asp Ser Ile Leu Arg Gln Leu
        1655 1660 1665
Asn Ser Phe His Ser Val Met Cys Gln His Gly Met Asp Pro Glu
        1670 1675 1680
Leu Ile Lys Gln Val Val Lys Gln Met Phe Tyr Ile Ile Gly Ala
        1685 1690 1695
Ile Thr Leu Asn Asn Leu Leu Leu Arg Lys Asp Met Cys Ser Trp
        1700 1705 1710
Ser Lys Gly Met Gln Ile Arg Tyr Asn Val Ser Gln Leu Glu Glu
        1715 1720 1725
Trp Leu Arg Asp Lys Asn Leu Met Asn Ser Gly Ala Lys Glu Thr
        1730 1735 1740
Leu Glu Pro Leu Ile Gln Ala Ala Gln Leu Leu Gln Val Lys Lys
        1745 1750 1755
Lys Thr Asp Asp Asp Ala Glu Ala Ile Cys Ser Met Cys Asn Ala
        1760 1765 1770
Leu Thr Thr Ala Gln Ile Val Lys Val Leu Asn Leu Tyr Thr Pro
        1775 1780 1785
Val Asn Glu Phe Glu Glu Arg Val Ser Val Ser Phe Ile Arg Thr
        1790 1795 1800
Ile Gln Met Arg Leu Arg Asp Arg Lys Asp Ser Pro Gln Leu Leu
        1805 1810 1815
Met Asp Ala Lys His Ile Phe Pro Val Thr Phe Pro Phe Asn Pro
        1820 1825 1830
Ser Ser Leu Ala Leu Glu Thr Ile Gln Ile Pro Ala Ser Leu Gly
        1835 1840 1845
Leu Gly Phe Ile Ser Arg Val
        1850 1855 .
```

7. A biomarker, characterized in that it is an antibody produced by the MYO5A protein according to claim 1, determines whether there is antiphospholipid syndrome by detecting the presence of antibodies to MYO5A protein.

8. A reagent kit, characterized in that it is made of MYO5A protein, determines whether there is antiphospholipid syndrome by detecting the presence of antibodies to MYO5A protein.

9. The reagent kit according to claim 8, characterized in that the MYO5A protein or a partial sequence fragment thereof is selected as the core part, and the detection method is any one of ELISA, chemiluminescence or POCT.

10. The reagent kit according to claim 8, characterized in that the MYO5A protein is used as the core material, and the target molecule detected and identified by the reagent kit is the antibody produced by the MYO5A protein, including at least one of IgG, IgM or IgA. The samples detected include serum, plasma or tissue fluid.
